(19) United States
(12) Patent Application Publication
(10) Pub. No.: US 2023/0142331 A1
(43) Pub. Date: May 11, 2023

(54) BANDAGE, HOT-MELT ADHESIVE FOR PRODUCING A BANDAGE,USE AND METHOD FOR THE PRODUCTION OF A HOT-MELT ADHESIVE

(71) Applicant: artimelt AG, Sursee (CH)

(72) Inventors: Judith NYFFELER, Ruswil (CH); Nicole SENN, Unterkulm (CH)

(21) Appl. No.: 17/911,675

(22) PCT Filed: Mar. 9, 2021
(86) PCT No.: PCT/EP2021/055862

§ 371 Date: Sep. 15, 2022

(30) Foreign Application Priority Data
Mar. 16, 2020 (EP) 20163389.8

(51) Int. Cl. A61F 13/02

(52) U.S. Cl. CPC A61F 13/0206; A61F 2013/00668

(57) ABSTRACT

The invention relates to the technical field of cohesive bandages. The invention relates to a bandage comprising a flat web material as a substrate, both main sides of the substrate being coated at least partially with a hot-melt adhesive. The invention also relates to a hot-melt adhesive for producing a bandage, to the use of a hot-melt adhesive for producing bandages, and to a method for producing a bandage. The hot-melt adhesive is based on a first polyolefin having—a proportion of 1-butene-derived monomer units; a proportion of ethylene-derived monomer units; and the hot-melt adhesive further comprises: at least one tackifier consisting of hydrocarbon resin and/or of hydrogenated hydrocarbon resin. The hot-melt adhesive can also contain a second polyolefin, preferably an amorphous polypropylene homo- or copolymer.

This application is a National Stage completion of PCT/EP2021/055862 filed Mar. 9, 2021, which claims priority to European application serial no. 20163389.8 filed Mar. 16, 2020.

### FIELD OF THE INVENTION

The invention relates to the technical field of cohesive bandages. Cohesive bandages are self-adhering fixation bandages for the fixation of wound dressings. They are used as therapeutic and prophylactic compression, support or protective bandages in sports, orthopedics, surgery or everyday life.

### BACKGROUND OF THE INVENTION

Cohesive bandages must meet various requirements. They must adhere well to themselves, be non-slip, especially for use on parts of the body that move a lot, and be skin-friendly and permeable to air.

Traditionally, a flexible textile with natural rubber from latex was used to make cohesive bandages. However, natural rubber has allergenic properties that affect a significant portion of the population. Therefore, natural rubber was more and more replaced with synthetic rubber.

WO 2017/109210 discloses a bandage made of elastic flat sheet material as substrate, which is coated or impregnated on both sides with synthetic rubber (polyisoprene, polybutadiene, polychloroprene). In the example given, the cohesive adhesive consists of 1.4 polyisoprene (NATSYN 2200), which is provided with an antioxidant and applied in solution. However, the adhesion of the Natsyn used falls significantly short of that of natural rubber, which has a negative impact on wearing comfort but also on the performance of bandages, especially compression bandages. There is therefore a need for cohesive bandages that are low allergenic but behave similarly to natural rubber in terms of adhesion performance.

### SUMMARY OF THE INVENTION

It is the object of the present invention to overcome the above-described disadvantages of the prior art. In particular, to propose a cohesive bandage which provides reliable adhesion to itself, comparable to that of natural rubber, but without causing allergic reactions.

This task is solved by a bandage, a hot-melt adhesive for producing a bandage, and a use and a process for producing a hot-melt adhesive according to the independent patent claims.

The bandage according to the invention comprises a flat web material as substrate, wherein both main sides of the substrate are at least partially coated with a hot melt adhesive, in particular with a hot melt adhesive comprising a first polyolefin of:

a proportion of monomer units derived from 1-butene;
a proportion of monomer units derived from ethylene;

and wherein the hot melt adhesive further comprises:

at least one tackifier of hydrocarbon resin and/or of hydrogenated hydrocarbon resin.

A flat web material is understood to be a flexible, ribbon-shaped substrate. The main sides of the flat web material are the two opposite sides which have the greatest areal extent. When the bandage is applied to the human body during intended use, the inner main side faces toward the human body, while the outer main side faces away from the human body. The inner main side may rest directly on the human skin at least in sections, may rest on a wound dressing at least in sections, and/or may rest on the outer main side of the same bandage or the outer main side of a second bandage at least in sections. Typically, the bandage is wrapped several times around a body part, e.g. an extremity, whereby the contact sections of the inner bandage main sides on the outer bandage main sides (sealing surfaces) ensure or support the adhesion of the bandage assembly.

The invention is based on the idea that it is desirable if the main sides of the bandages have a high adhesion to each other and at the same time the main sides of the bandages have a particularly low adhesion to surfaces other than the main sides of the bandages (e.g. to the skin or clothing of a patient). Adhesion is understood to mean, in particular, the cutting off of a material in the T-Peel test.

Usually, adhesion is represented as the interplay of adhesion, cohesion and tack. Tack refers to the speed at which an adhesive bond is formed, and adhesion to the bond strength achieved between the adhesive and the surface to be bonded (physical, macroscopically observable attraction). Adhesives are often designed to have high tack and adhesion, but this can be directed against a variety of partner surfaces. This is not desirable in the case of a cohesive bandage. In particular, the cohesive bandage should not adhere to the skin to ensure painless removal of the bandage. Nor should the cohesive bandage adhere to other surfaces, such as clothing worn over the bandage. Such adhesion would compete with the adhesion of the bandage to itself and exert forces that cause the bandage to slip or detach.

The bandage according to the invention takes this problem into account. The first polyolefin used ensures a high adhesive strength when the main side is directed against the main side, but does not form any interfering adhesive bonds with other surfaces. The detailed values are given below. Due to the hydrocarbon resin and/or hydrogenated hydrocarbon resin tackifier(s) used, the desired adhesive bond between two main sides of the substrate is also quickly established without the need for excessive pressure. The use of adhesive strips or clamps can be dispensed with thanks to the properties of the bandage according to the invention.

One aspect of the invention relates to a bandage as described above, wherein the hot melt adhesive comprises at least a second polyolefin, wherein the second polyolefin is preferably amorphous, more preferably propylene-based, more preferably a blend of propylene homopolymer and propylene-based copolymer. For example, the second polyolefin may be a blend of a propylene homopolymer and a copolymer comprising monomer units derived from propylene and monomer units derived from ethylene. Such a second polyolefin preferably has a low molecular weight. A typical melt viscosity is from 1500 to 2500 mPa·s, preferably from 1800 to 2200 mPa·s (at 190° C.) Such a second polyolefin is soft and tacky and thus additionally improves the flexibility, but also the adhesion properties of the bandage.

It is preferred that the combined proportions of the first and second polyolefins in the hot melt adhesive is total 17 to 43 wt %, preferably 20 to 42 wt %, particularly preferably 25 to 40 wt %. Such a polyolefin content optimizes the previously described adhesion strength of the cohesive bond to itself. It is preferred, that in this case, the proportion of the first polyolefin from a proportion of monomer units derived from 1-butene and from a proportion of monomer units derived from ethylene is 8 to 25 wt. %, preferably 10 to 20 wt. %, of the total weight of the hot melt adhesive.

One aspect of the invention relates to a bandage as described above, wherein in the hot melt adhesive the proportionate ratio (in weight percent) of tackifier to total polyolefin is in the range of 0.6 to 3, preferably in the range of 0.8 to 1.8, more preferably in the range of 1 to 1.4.

It is preferred if the hot melt adhesive comprises a lubricant additive, preferably a wax, more preferably a polypropylene wax. The relative proportion of the lubricant additive to the total mass of the hot melt adhesive is preferably at least 10% by weight, more preferably 15-40% by weight and even more preferably 20-35% by weight.

Since the hotmelt adhesive is applied to a flexible, in particular stretchable, substrate to produce the bandage, viscosity must be ensured both in the application step and in the finished product. This is ensured by the use of a relatively high lubricant content.

It is preferred that the proportion of 1-butene derived monomer units in the first polyolefin is 80 to 92 wt %, preferably 84 to 90 wt %. It is particularly preferred that the copolymer is a butene-1/ethylene copolymer, such that the ethylene monomer content complements the 1-butene monomer content to 100%. It is preferred that the first polyolefin has a melt flow rate (MFR) of 0.5 to 7 g/10 min, preferably 1.0 to 3 g/10min, particularly preferably about 1.3 g/10 min, measurable according to DIN EN ISO 1133-1:2012-03 (190° C.; 2.16 kg).

A surface coated with such a first polyolefin has a particularly high adhesion to a partner surface coated with the same first polyolefin (sealing surfaces). The two partner surfaces can be easily peeled off from each other (by hand), but can also be easily rejoined. They have a nearly constant peel strength, even with repeated use. The term “peelable” refers to a separation at the interface of the sealing surface. It is a particular advantage of such a first polyolefin that it exhibits the advantageous properties, i.e. peelability and adhesion of mating surfaces to each other, at room temperature (22° C.±5° C.)

It is preferred that the hot melt adhesive has a melt viscosity of 1,000 to 50,000 mPa·s, preferably 10,000 to 45,000 mPa·s, particularly preferably 15,000 to 30,000 mPa·s, measurable according to EN ISO 3219 (Cone: 35 mm/2°; Speed=31.6 sec⁻¹; 160° C.). A hotmelt adhesive having this melt viscosity is particularly easy to compound and can be applied to the substrate without problems. The viscosity is important for uniform, easy and yet well adhering application. If the hotmelt adhesive were to have an excessively high or excessively low viscosity, the viscosity would still have to be adjusted by means of solvent additives to allow conventional application (spray application, printing, dipping, knife or roller doctoring or casting processes). Especially if organic solvents are used, such a formulation pollutes the environment and requires measures to ensure occupational safety. In addition, there is then a risk that the anchoring of the material in the substrate, especially a textile substrate, will be inadequate. If the viscosity is too high, a more complex process using nozzle extrusion becomes necessary. A hot melt adhesive, however, which has a melt viscosity as described above, avoids these problems and, despite the absence of solvents, can be applied well through slot dies directly to the substrate where it anchors very well.

It is preferred that the first polyolefin has a glass transition temperature of −27° C., measurable by DMTA analysis. A polyolefin having such a glass transition temperature has sufficient crystalline or amorphous fractions at room temperature so that the required tack is achieved in the finished product.

In DMTA (dynamic mechanical thermal analysis), cast specimens of 76 mm×13 mm×1 mm are fixed in the DMTA machine for exerting tensile stress. The frequency of tension and relaxation of the specimen is set to 1 Hz. The DMTA transfers the elastic response of the specimen from −100° C. to 130° C. In this way, it is possible to plot the elastic response versus temperature. The elastic modulus for a viscoelastic material is defined as E=E′+iE″. The DMTA can split the two components E′ and E″ based on their resonance and plot E′ versus temperature. Similarly, E′+E″=tan(δ) can be plotted against temperature. The glass transition temperature Tg is read as the temperature at the maximum of the E′+E″=tan(δ)versus temperature curve.

One aspect of the invention relates to a bandage as described above, wherein the hot melt adhesive has a film strength of from 0.5 to 1.5 N/mm², preferably from 0.6 to 1.1 N/mm², measurable according to DIN 53504. Another aspect of the invention relates to a bandage as described above, wherein the hot melt adhesive has an elongation at break of from 700 to 1200%, preferably an elongation at break of from 800 to 1000%, measurable according to DIN 53504. A bandage which has a high film strength and elongation at break is particularly suitable for the manufacture of cohesive bandages, since in this way the forces acting on the bandage in everyday use do not impair the integrity of the bandage. Such a bandage has high extensibility. The hot melt adhesive applied to an elastic textile flat web material, for example, can expand and contract together with the substrate without cracking, which is particularly advantageous for cohesive bandages.

As already described at the beginning, the bandage should be skin-friendly and permeable to air and, if necessary, be able to exert a compression effect on the body part to be treated. The substrate can therefore be, for example, a woven fabric, knitted fabric, knitted fabric or a non-woven fabric. The substrate of the bandage as described above is preferably a woven or non-woven textile, preferably a woven or non-woven textile that is elastic in the longitudinal and/or transverse direction. For example, an elastic fabric construction may result from combining permanently elastic elastane yarns with rigid cotton yarns.

Another aspect of the invention relates to a hot melt adhesive for making a bandage as described above, wherein the hot melt adhesive is based on a first polyolefin, comprising
a proportion of monomer units derived from 1-butene;
a proportion of monomer units derived from ethylene;
and wherein the hot melt adhesive further comprises:
at least one tackifier of hydrocarbon resin and/or of hydrogenated hydrocarbon resin;
wherein the peel strength at room temperature of a composite of two substrates coated with the hotmelt adhesive is at least 1 to 10 N/25 mm, preferably 1.5 to 6.5 N/25 mm, particularly preferably 4 to 6 N/25 mm, measurable according to ASTM D 1876-01.

Unless explicitly described otherwise, the specifications of measuring method ASTM D 1876-01 are applicable. Any deviations can be seen in the following examples (Example 3). A hotmelt adhesive having the said peel strength is suitable for the manufacture of a bandage which can be used as a protective, wound or compression bandage without the use of aids, i.e. without the use of e.g. adhesive strips or staples. The hold of the protective, wound or compression bandage is ensured to a sufficient extent by the adhesion of the sealing surfaces.

It is preferred if the hot melt adhesive is such that the peel strength of a bond from a substrate coated with the hot melt adhesive to materials not coated with the hot melt adhesive is minimal to non-existent at room temperature. This can be assessed with qualitative testing. As can be seen from the following specific embodiments (Example 4), the adhesion of adhesive-coated films and textiles to the skin of test subjects can be subjectively evaluated after a certain time has elapsed, and the result compared with that for conventional adhesives.

Another aspect of the invention relates to the use of a hot melt adhesive as described above for the manufacture of bandages. The hot melt adhesive is based on a first polyolefin, in particular comprising

a proportion of 1-butene-derived monomer units;

a proportion of monomer units derived from ethylene;

wherein the hot melt adhesive further comprises:

at least one tackifier of hydrocarbon resin and/or hydrogenated hydrocarbon resin.

It is preferred that the hot melt adhesive is composed as described above and that the hot melt adhesive or the films and bandages made from it have the properties described above.

Another aspect of the invention relates to a method of making a bandage, comprising the following steps:

Providing a flat web material as substrate, in particular a woven or non-woven textile elastic in longitudinal and/or transverse direction; and

Applying a hot melt adhesive to both opposing major sides of a substrate.

The double-sided application of the hotmelt adhesive can be carried out by extruding, laminating, etc. The hotmelt adhesive can also be applied to both sides. It is preferred if the hotmelt adhesive is provided solvent-free. “Solvent-free” is understood to mean the absence of aqueous phases or organic phases, in particular alcohols, ethers, aromatics, etc. It is preferred if the hotmelt adhesive is extruded onto both sides of the flat web material by means of extrusion through wide-slit nozzles.

### BRIEF DESCRIPTION OF THE DRAWING

The invention is explained in more detail below in examples of embodiments and with reference to figures, without limiting the invention to these specific examples of embodiments.

It shows:

FIG. 1 Diagram of the results from the qualitative skin adhesion test according to example 4

### DETAILED DESCRIPTION OF THE PREFERRED EMBODIMENTS

### EXAMPLE 1

A hot melt adhesive is mixed from the following ingredients:

| | Proportion |
|---|---|
| | (% by weight) |
| Compounded butene-1/ethylene co-polymer (72 wt % | 15 |
| butene-1; 13 wt % ethylene; 15 wt % tackifier). | |
| Amorphous polyolefin (blend of propylene homopolymer | 20 |
| and propylene-ethylene copolymer, low molecular | |
| weight) | |
| Hydrocarbon resin (cycloaliphatic hydrocarbon resin, | 35 |
| amorphous, low molecular weight, water bright) | |
| Polypropylene wax (low molecular weight, from | 28 |
| metallocene catalysis) | |
| Stabilizer | 2 |

### EXAMPLE 2

The melt viscosity of the adhesive according to Example 1 was determined according to EN ISO 3219 and was 22′000 mPa·s. The film strength of the adhesive was measured according to DIN 53504 and was 0.9 N/mm. The elongation at break of the adhesive was also determined according to DIN 53504 and was 900%.

### EXAMPLE 3

A peel strength test was performed according to ASTM D 1876-01. An untreated 23my PET film was used as the substrate. The composition from Example 1 was layered directly onto a PET substrate through wide slot nozzles. The thickness of the adhesive layer was 12-15 g/cm². The coated substrates were stored for 24 h at 23° C. and 50% relative humidity.

The specimens were bonded with the adhesive sides facing each other and lightly pressed. A test strip of 100×25 mm was cut from the composite using a Japanese knife and rolled twice in both directions with a weight of 2 kg. The test strip was then stored for another 20 min. The thickness of the resulting adhesive layer after the bonding step was 24 to 30 g/m². At least 4 sample strips are produced for each sample material. The T-peel measurement was performed on a tensile testing machine from Instron, with the gripper arms set to a separation speed of 300 mm/min. The force was measured by the tensile testing machine over the entire pull-off distance of 100 mm and the arithmetic mean value over the measured distance (minus the first and last 10% of the distance) was calculated.

The maximum, minimum, and average forces for a single test strip were 8N/25 mm, 2N/25 mm, and 5N/25 mm, respectively. The average force per section averaged over all specimens was 5N/25 mm.

### EXAMPLE 4

A qualitative assessment of the adhesion of the hot melt adhesive and the bandage coated with the hot melt adhesive to human skin was performed on 20 test subjects. The adhesives and substrates measured are shown in the table below:

| Label | Adhesive | Substrate | Coating | |
|---|---|---|---|---|
| M11.159 | Hotmelt PPE, based on | PET 23my film | 25 | gsm |
| | thermoplastic rubber, | | | |
| | with high peel strength | | | |
| M11.180 | Hotmelt PPE based on | PET 23my film | 25 | gsm |
| | thermoplastic rubber | | | |
| | with low peel strength | | | |
| M11.1506 | Hotmelt PSA, acrylate- | PET 23my film | 100 | gsm |
| | based with weak to | | | |
| | medium peel strength | | | |
| M13.1048 | Hotmelt PPE based on | PET 23my film | 25 | gsm |
| | thermoplastic rubber | | | |
| | with medium peel | | | |
| | strength | | | |
| Ex. 1, | Based on 1-butene/ | PET 23my film | 12-15 | gsm |
| smooth | ethylene co-polymer | | | |
| carrier | | | | |
| Ex. 1, | Based on 1-butene/ | Elastic fabric | 12-15 | gsm |
| textile | ethylene co-polymer | made of cotton | | |
| carrier | | and elastane | | |

The test strips were each coated with the adhesive using a wide slit nozzle, and the coated substrates were stored for 24 h at 23° C. and 50% relative humidity.

Then, one test strip per adhesive was applied to the forearm of each of the 20 test subjects under manual pressure and the test strips were left on the skin for 20 min. At the end of the 20 min, the 6 test strips were sequentially peeled off the skin of the test subjects and the test subjects qualitatively assessed the adhesion according to the following scale: 5—Strongest adhesion; 4—Significant adhesion; 3—Medium adhesion; 2—Light adhesion; 1—Minimal adhesion; 0—No adhesion.

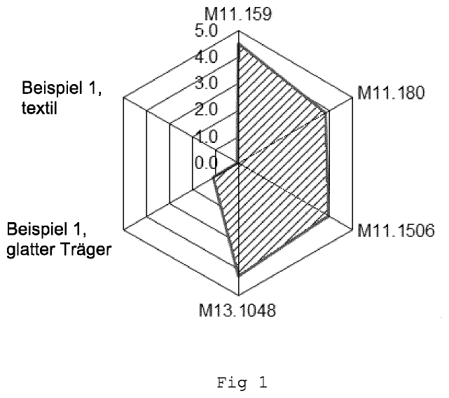

The results of the tests are shown in FIG. 1. From the tests, it can be seen that the hotmelt adhesive according to Example 1 still shows a low residual adhesion when coated onto a smooth backing. However, in none of the 20 test subjects did the hotmelt adhesive coated on bandage material show adhesion.

1. A bandage comprising a flat web material as a substrate, both main sides of the substrate being at least partially coated with a hot melt adhesive comprising a first polyolefin of:
a proportion of monomer units derived from of 1-butene; and
a proportion of monomer units derived from ethylene;
and wherein the hot melt adhesive further comprises:
at least one tackifier of hydrocarbon resin and/or of hydrogenated hydrocarbon resin.

2. A bandage of claim 1, wherein the hot melt adhesive comprises at least a second polyolefin.

3. A bandage of claim 2, wherein the combined proportions of the first and second polyolefins in the hot melt adhesive is from 17 to 43 wt %.

4. A bandage according to claim 1, wherein in the hot melt adhesive the proportionate ratio (in wt %) of tackifier to total polyolefin is in the range of 0.6 to 3.

5. A bandage according to claim 1, wherein the hot melt adhesive further comprises a lubricant additive.

6. The bandage of claim 5, wherein the relative proportion of the lubricant additive is at least 5% by weight.

7. The bandage according to claim 1, wherein the proportion of 1-butene derived monomer units in the first polyolefin is 80 to 92 wt %.

8. The bandage according to claim 1, wherein the hot melt adhesive has a melt viscosity of 1,000 to 50,000 mPa·s, measurable according to EN ISO 3219.

9. The bandage according to claim 1, wherein the hot melt adhesive has a film strength of 0.5 to 1.5 N/mm², according to DIN 53504.

10. The bandage according to claim 1, wherein the hot melt adhesive has an elongation at break of 700 to 1200%, measurable according to DIN 53504.

11. The bandage according to claim 1, wherein the substrate is a woven or nonwoven textile.

12. A hot melt adhesive for making a bandage according to claim 1, wherein the hot melt adhesive is based on a first polyolefin, comprising
a proportion of monomer units derived from 1-butene;
a proportion of monomer units derived from ethylene;
and wherein the hot melt adhesive further comprises:
at least one tackifier selected from at least one of a hydrocarbon resin and a hydrogenated hydrocarbon resin;
wherein the peel strength at room temperature of a composite of two substrates coated with the hotmelt adhesive is at least 1 to 10 N/25 mm, measurable according to ASTM D 1876-01.

13. A method of manufacturing bandages comprising applying a hot melt adhesive to a substrate, the hot melt adhesive based on a first polyolefin, the first polyolefin comprising:
a proportion of monomer units derived from 1-butene; and
a proportion of monomer units derived from ethylene;
wherein the hot melt adhesive further comprises at least one tackifier comprising hydrocarbon resin and/or hydrogenated hydrocarbon resin.

14. A method of manufacturing a bandage comprising the following steps:
Providing a flat web material as substrate;
providing a hot melt adhesive according to claim 12; and
double-sided application of the hotmelt adhesive to the substrate.

15. The method of claim 14, wherein the hot melt adhesive is provided solvent-free.

16. The bandage according to claim 2, wherein the second polyolefin is amorphous.

17. The bandage according to claim 16, wherein the second polyolefin is propylene-based.

18. The bandage according to claim 17, wherein the second polyolefin is a blend of propylene homopolymer and propylene-based copolymer.

19. The bandage according to claim 5, wherein the lubricant is a wax.

20. The bandage according to claim 11, wherein the woven or nonwoven textile is elastic in a longitudinal and/or a transverse direction.
